(19) United States
(12) Patent Application Publication
(10) Pub. No.: US 2009/0149772 A1
(43) Pub. Date: Jun. 11, 2009

(54) Temperature Indicator for Cooling Products

(75) Inventors: J. Gavin MacDonald, Decatur, GA (US); Kaiyuan Yang, Cumming, GA (US); Kelly D. Arehart, Roswell, GA (US)

Correspondence Address: DORITY & MANNING, P.A., POST OFFICE BOX 1449, GREENVILLE, SC, 29602-1449, US

(73) Assignee: KIMBERLY-CLARK WORLDWIDE, INC., Neenah, WI (US)

(21) Appl. No.: 11/950,824

(22) Filed: Dec. 5, 2007

(51) Int. Cl. A61B 5/01; B32B 5/16; C09K 5/00; D04H 13/00

(52) U.S. Cl. 600/549; 252/71; 428/323; 442/327

(57) ABSTRACT

A cooling product (e.g., mask, glove, sock, etc.) configured to provide a cooling effect to the body part of a user is provided. The cooling product contains a thermochromic composition that undergoes a color change at a certain temperature. The color change may signal to a user that the cooling product is cold, thus providing an indication that the desired treatment is still functioning. Likewise, the color change may signal that the product is warm, thus providing an indication that the treatment is complete.

### BACKGROUND OF THE INVENTION

Cooling products are used for a wide variety of purposes, such as for cooling a body part (e.g., forehead, cheek, jaws, etc.) of a person who is feverish, injured, etc. Simple ice packs, for instance, are often used to help reduce swelling. However, ice packs do not normally permit compression on and around the injured area so as to achieve the best possible minimization of swelling. Further, when an ice pack is applied, the injured person has little freedom of movement. In an attempt to overcome these problems, wraps have been developed that are more flexible in nature. One example of such a wrap is described in U.S. Pat. No. 4,377,160 to Romaine. More specifically, this wrap contains a gel-like material formed by gelling a polyvinyl alcohol solution. A sheet or strip of thin polyurethane foam is dipped in the polyvinyl alcohol solution and thereafter dipped in a reactive gelling agent solution, such as an aqueous borax solution, to form a gel. Unfortunately, however, it is often difficult to readily detect when the wrap is cool enough to begin treatment, and conversely when the wrap begins to warm near the completion of treatment.

As such, a need currently exists for a technique for simply and rapidly detecting the temperature of a cooling product.

### SUMMARY OF THE INVENTION

In accordance with one embodiment of the present invention, a cooling product for providing treatment to the body part of a user is disclosed. The cooling product comprises a gel configured to cool the skin of a body part for a certain time period, the time period defining a cooling cycle. The cooling product further comprises a thermochromic composition that possesses a first color during the cooling cycle and a second color after completion of the cooling cycle, the first color being visually distinguishable from the second color.

In accordance with another embodiment of the present invention, a method for monitoring the degree of cooling being provided to a body part of a user is disclosed. The method comprises providing a cooling product that comprises a gel configured to cool the skin of a body part for a certain time period, the time period defining a cooling cycle. The cooling product further comprising a thermochromic composition that possesses a first color during the cooling cycle and a second color after completion of the cooling cycle. The cooling product is placed adjacent to a body part. The thermochromic composition is observed, wherein observation of the first color indicates that the gel is cooling the skin during the cooling cycle and observation of the second color indicates that the cooling cycle is complete.

Other features and aspects of the present invention are set forth in greater detail below.

### BRIEF DESCRIPTION OF THE DRAWINGS

A full and enabling disclosure of the present invention, including the best mode thereof, directed to one of ordinary skill in the art, is set forth more particularly in the remainder of the specification, which makes reference to the appended figures in which:

FIG. 1 is a perspective view of one embodiment of a cooling product of the present invention;

FIG. 2 is a plan view of the cooling product illustrated in FIG. 1; and

FIG. 3 is a schematic illustration of another embodiment of a cooling product of the present invention.

Repeat use of reference characters in the present specification and drawings is intended to represent same or analogous features or elements of the present disclosure.

### DETAILED DESCRIPTION OF REPRESENTATIVE EMBODIMENTS

### Definitions

As used herein the term “nonwoven” web or layer means a web having a structure of individual fibers or threads which are interlaid, but not in an identifiable manner as in a knitted fabric. Nonwoven webs may include, for instance, meltblown webs, spunbond webs, airlaid webs, carded webs, hydraulically entangled webs, etc. The basis weight of a nonwoven web may vary, such as from about 5 grams per square meter (“gsm”) to 150 gsm, in some embodiments from about 10 gsm to about 1000 gsm, and in some embodiments, from about 15 gsm to about 70 gsm.

As used herein, the term “meltblown web” generally refers to a nonwoven web that is formed by a process in which a molten thermoplastic material is extruded through a plurality of fine, usually circular, die capillaries as molten fibers into converging high velocity gas (e.g., air) streams that attenuate the fibers of molten thermoplastic material to reduce their diameter, which may be to microfiber diameter. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Buntin, et al., which is incorporated herein in its entirety by reference thereto for all purposes. Generally speaking, meltblown fibers may be microfibers that are substantially continuous or discontinuous, generally smaller than 10 micrometers in diameter, and generally tacky when deposited onto a collecting surface.

As used herein, the term “spunbond web” generally refers to a web containing small diameter substantially continuous fibers. The fibers are formed by extruding a molten thermoplastic material from a plurality of fine, usually circular, capillaries of a spinnerette with the diameter of the extruded fibers then being rapidly reduced as by, for example, eductive drawing and/or other well-known spunbonding mechanisms. The production of spunbond webs is described and illustrated, for example, in U.S. Pat. No. 4,340,563 to Appel, et al., U.S. Pat. No. 3,692,618 to Dorschner, et al., U.S. Pat. No. 3,802,817 to Matsuki, et al., U.S. Pat. No. 3,338,992 to Kinney, U.S. Pat. No. 3,341,394 to Kinney, U.S. Pat. No. 3,502,763 to Hartman, U.S. Pat. No. 3,502,538 to Levy, U.S. Pat. No. 3,542,615 to Dobo, et al., and U.S. Pat. No. 5,382,400 to Pike, et al., which are incorporated herein in their entirety by reference thereto for all purposes. Spunbond fibers are generally not tacky when they are deposited onto a collecting surface. Spunbond fibers may sometimes have diameters less than about 40 micrometers, and are often between about 5 to about 20 micrometers.

As used herein, the term “coform” generally refers to a thermal composite material that contains a mixture or stabilized matrix of thermoplastic fibers and a second non-thermoplastic material. As an example, coform materials may be made by a process in which at least one meltblown die head is arranged near a chute through which other materials are added to the web while it is forming. Such other materials may include, but are not limited to, fibrous organic materials such as woody or non-woody pulp such as cotton, rayon, recycled paper, pulp fluff and also superabsorbent particles, inorganic and/or organic absorbent materials, treated polymeric staple fibers and so forth. Some examples of such coform materials are disclosed in U.S. Pat. No. 4,100,324 to Anderson, et al.; U.S. Pat. No. 5,284,703 to Everhart, et al.; and U.S. Pat. No. 5,350,624 to Georger, et al.; which are incorporated herein in their entirety by reference thereto for all purposes.

### DETAILED DESCRIPTION

Reference now will be made in detail to various embodiments of the invention, one or more examples of which are set forth below. Each example is provided by way of explanation, not limitation of the invention. In fact, it will be apparent to those skilled in the art that various modifications and variations may be made in the present invention without departing from the scope or spirit of the invention. For instance, features illustrated or described as part of one embodiment, may be used on another embodiment to yield a still further embodiment. Thus, it is intended that the present invention cover such modifications and variations.

Generally speaking, the present invention is directed to a cooling product (e.g., mask, glove, sock, etc.) configured to provide a cooling effect to the body part of a user. The cooling product contains a thermochromic composition that undergoes a color change at a certain temperature. The color change may signal to a user that the cooling product is cold, thus providing an indication that the desired treatment is still functioning. Likewise, the color change may signal that the product is warm, thus providing an indication that the treatment is complete.

Any thermochromic substance may generally be employed in the present invention. For example, liquid crystals may be employed as a thermochromic substance in some embodiments. The wavelength of light (“color”) reflected by liquid crystals depends in part on the pitch of the helical structure of the liquid crystal molecules. Because the length of this pitch varies with temperature, the color of the liquid crystals is also a function of temperature. One particular type of liquid crystal that may be used in the present invention is a liquid crystal cholesterol derivative. Exemplary liquid crystal cholesterol derivatives may include alkanoic and aralkanoic acid esters of cholesterol, alkyl esters of cholesterol carbonate, cholesterol chloride, cholesterol bromide, cholesterol acetate, cholesterol oleate, cholesterol caprylate, cholesterol oleyl-carbonate, and so forth. Other suitable liquid crystal cholesterol derivatives are described in U.S. Pat. No. 3,600,060 to Churchill, et al.; U.S. Pat. No. 3,619,254 to Davis; and U.S. Pat. No. 4,022,706 to Davis, which are incorporated herein in their entirety by reference thereto for all purposes.

In addition to liquid crystals, another suitable thermochromic substance that may be employed in the present invention is a proton accepting chromogen (“Lewis base”). In solution, the protonated form of the chromogen predominates at acidic pH levels (e.g., pH of about 4 or less). When the solution is made more alkaline through protonation, however, a color change occurs. One particularly suitable class of proton-accepting chromogens are leuco dyes, such as phthalides; phthalanes; acyl-leucomethylene compounds; fluoranes; spiropyranes; cumarins; and so forth. Exemplary fluoranes include, for instance, 3,3′-dimethoxyfluorane, 3,6-dimethoxyfluorane, 3,6-di-butoxyfluorane, 3-chloro-6-phenylamino-flourane, 3-diethylamino-6-dimethylfluorane, 3-diethylamino-6-methyl-7-chlorofluorane, and 3-diethyl-7,8-benzofluorane, 3,3′-bis-(p-dimethyl-aminophenyl)-7-phenylaminofluorane, 3-diethylamino-6-methyl-7-phenylamino-fluorane, 3-diethylamino-7-phenyl-aminofluorane, and 2-anilino-3-methyl-6-diethylamino-fluorane. Likewise, exemplary phthalides include 3,3′,3″-tris(p-dimethylamino-phenyl)phthalide, 3,3′-bis(p-dimethyl-aminophenyl)phthalide, 3,3-bis(p-diethylamino-phenyl)-6-dimethylamino-phthalide, 3-(4-diethylaminophenyl)-3-(1-ethyl-2-methylindol-3-yl)phthalide, and 3-(4-diethylamino-2-methyl)phenyl-3-(1,2-dimethylindol-3-yl)phthalide. Still other suitable chromogens are described in U.S. Pat. No. 4,620,941 to Yoshikawa, et al.; U.S. Pat. No. 5,281,570 to Hasegawa, et al.; U.S. Pat. No. 5,350,634 to Sumii, et al.; and U.S. Pat. No. 5,527,385 to Sumii, et al., which are incorporated herein in there entirety for all purposes.

A desensitizer may also be employed in conjunction with the proton-accepting chromogen to facilitate protonation at the desired temperature. More specifically, at a temperature below the melting point of the desensitizer, the chromogen generally possesses a first color (e.g., white). When the desensitizer is heated to its melting temperature, the chromogen becomes protonated, thereby resulting in a shift of the absorption maxima of the chromogen towards either the red (“bathochromic shift”) or blue end of the spectrum (“hypsochromic shift”). The nature of the color change depends on a variety of factors, including the type of proton-accepting chromogen utilized and the presence of any additional temperature-insensitive chromogens. The color change is typically reversible in that the chromogen deprotonates when cooled. Although any desensitizer may generally be employed in the present invention, it is typically desired that the desensitizer have a low volatility. For example, the desensitizer may have a boiling point of about 150° C. or higher, and in some embodiments, from about 170° C. to 280° C. Likewise, the melting temperature of the desensitizer is also typically from about −5° C. to about 45° C., in some embodiments from about 0° C. to about 40° C., and in some embodiments from about 2° C. to about 34° C. Examples of suitable desensitizers may include saturated or unsaturated alcohols containing about 6 to 30 carbon atoms, such as octyl alcohol, dodecyl alcohol, lauryl alcohol, cetyl alcohol, myristyl alcohol, stearyl alcohol, behenyl alcohol, geraniol, etc.; esters of saturated or unsaturated alcohols containing about 6 to 30 carbon atoms, such as butyl stearate, lauryl laurate, lauryl stearate, stearyl laurate, methyl myristate, decyl myristate, lauryl myristate, butyl stearate, lauryl palmitate, decyl palmitate, palmitic acid glyceride, etc.; azomethines, such as benzylideneaniline, benzylidenelaurylamide, o-methoxybenzylidene laurylamine, benzylidene p-toluidine, p-cumylbenzylidene, etc.; amides, such as acetamide, stearamide, etc.; and so forth.

The thermochromic composition may also include a proton-donating agent (also referred to as a “color developer”) to facilitate the reversibility of the color change. Such proton-donating agents may include, for instance, phenols, azoles, organic acids, esters of organic acids, and salts of organic acids. Exemplary phenols may include phenylphenol, bisphenol A, cresol, resorcinol, chlorolucinol, β-naphthol, 1,5-dihydroxynaphthalene, pyrocatechol, pyrogallol, trimer of p-chlorophenol-formaldehyde condensate, etc. Exemplary azoles may include benzotriaoles, such as 5-chlorobenzotriazole, 4-laurylaminosulfobenzotriazole, 5-butylbenzotriazole, dibenzotriazole, 2-oxybenzotriazole, 5-ethoxycarbonylbenzotriazole, etc.; imidazoles, such as oxybenzimidazole, etc.; tetrazoles; and so forth. Exemplary organic acids may include aromatic carboxylic acids, such as salicylic acid, methylenebissalicylic acid, resorcylic acid, gallic acid, benzoic acid, p-oxybenzoic acid, pyromellitic acid, β-naphthoic acid, tannic acid, toluic acid, trimellitic acid, phthalic acid, terephthalic acid, anthranilic acid, etc.; aliphatic carboxylic acids, such as stearic acid, 1,2-hydroxystearic acid, tartaric acid, citric acid, oxalic acid, lauric acid, etc.; and so forth. Exemplary esters may include alkyl esters of aromatic carboxylic acids in which the alkyl moiety has 1 to 6 carbon atoms, such as butyl gallate, ethyl p-hydroxybenzoate, methyl salicylate, etc.

If desired, one or more of the above-described components may be encapsulated to enhance the stability of the thermochromic substance during use. For example, a chromogen, desensitizer, developer, etc. may be mixed with a polymer resin (e.g., thermoset) according to any conventional method, such as interfacial polymerization, in-situ polymerization, etc. Suitable thermoset resins may include, for example, polyester resins, polyurethane resins, melamine resins, epoxy resins, diallyl phthalate resins, vinylester resins, and so forth. The resulting mixture may then be granulated and optionally coated with a hydrophilic macromolecular compound, such as alginic acid and salts thereof, carrageenan, pectin, gelatin and the like, semisynthetic macromolecular compounds such as methylcellulose, cationized starch, carboxymethylcellulose, carboxymethylated starch, vinyl polymers (e.g., polyvinyl alcohol), polyvinylpyrrolidone, polyacrylic acid, polyacrylamide, maleic acid copolymers, and so forth. The resulting microcapsules typically have a mean particle size of from about 5 nanometers to about 25 micrometers, in some embodiments from about 10 nanometers to about 10 micrometers, and in some embodiments, from about 50 nanometers to about 5 micrometers. Various other suitable encapsulation techniques are also described in U.S. Pat. No. 4,957,949 to Kamada, et al.; U.S. Pat. No. 5,431,697 to Kamata, et al.; and U.S. Pat. No. 6,863,720 to Kitagawa, et al., which are incorporated herein in their entirety by reference thereto for all purposes.

The amount of the polymer resin(s) (e.g., thermoset) used to form such color-changing microcapsules may vary, but is typically from about 20 wt. % to about 80 wt. %, in some embodiments from about 30 wt. % to about 70 wt. %, and in some embodiments, from about 40 wt. % to about 60 wt. % of the microcapsules. The amount of the proton-accepting chromogen(s) employed may be from about 0.1 wt. % to about 20 wt. %, in some embodiments from about 0.5 wt. % to about 15 wt. %, and in some embodiments, from about 1 to about 10 wt. % of the microcapsules. The proton-donating agent(s) may constitute from about 0.5 to about 30 wt. %, in some embodiments from about 1 wt. % to about 20 wt. %, and in some embodiments, from about 2 wt. % to about 15 wt. % of the microcapsules. In addition, the desensitizer(s) may constitute from about 10 wt. % to about 70 wt. %, in some embodiments from about 15 wt. % to about 60 wt. %, and in some embodiments, from about 20 wt. % to about 50 wt. % of the microcapsules.

The nature and weight percentage of the components used in the color-changing composition are generally selected so that it changes from one color to another color, from no color to a color, or from a color to no color at a desired activation temperature. The desired activation temperature depends largely on the specific nature of the cooling product. Cooling products, for example, are generally designed to reach a reduced temperature of from about 20° C. to about 45° C., in some embodiments from about 22° C. to about 40° C., and in some embodiments from about 24° C. to about 34° C. Thus, the thermochromic composition may have an activation temperature within the ranges noted above so that it possesses one color when the product is providing cooling, and another color when the treatment is complete and the product begins to warm. Commercially available thermochromic substances that have an activation temperature with the desired ranges may be obtained from Matsui Shikiso Chemical Co., Ltd. of Kyoto, Japan under the designation “Chromicolor” (e.g., Chromicolor AQ-Ink) or from Color Change Corporation of Streamwood, Ill. (e.g., black leuco powder having a transition of 33° C., red leuco powder having a transition of 28° C., yellow and red leuco powder having a transition of 31° C., or blue leuco powder having a transition of 33° C. or 36° C.).

The thermochromic composition of the present invention is applied to the cooling product so that it is visible during use. For example, the composition may be coated onto one or more surfaces of a substrate (e.g., nonwoven web, woven fabric, knit fabric, paper web, film, foam, etc.) of the cooling product using any known technique, such as printing, dipping, spraying, melt extruding, coating (e.g., solvent coating, powder coating, brush coating, etc.), and so forth. The thermochromic composition may cover an entire surface of the cooling product, or may only cover a portion of the surface. For instance, to maintain absorbency, porosity, flexibility, and/or some other characteristic of the cooling product, it may sometimes be desired to apply the thermochromic composition so as to cover less than 100%, in some embodiments from about 10% to about 80%, and in some embodiments, from about 20% to about 60% of the area of one or more surfaces of the cooling product. The thermochromic composition may, for example, be applied to the cooling product in a preselected pattern (e.g., reticular pattern, diamond-shaped grid, dots, and so forth). It should be understood, however, that the coating may also be applied uniformly to one or more surfaces of the cooling product.

If desired, the thermochromic composition may also be applied to a separate substrate (e.g., strip) that is subsequently adhered or otherwise attached to the cooling product. For example, the strip may contain a facestock material commonly employed in the manufacture of labels, such as paper, polyester, polyethylene, polypropylene, polybutylene, polyamides, etc. An adhesive, such as a pressure-sensitive adhesive, heat-activated adhesive, hot melt adhesive, etc., may be employed on one or more surfaces of the facestock material to help adhere it to a surface of the substrate. Suitable examples of pressure-sensitive adhesives include, for instance, acrylic-based adhesives and elastomeric adhesives. In one embodiment, the pressure-sensitive adhesive is based on copolymers of acrylic acid esters (e.g., 2-ethyl hexyl acrylate) with polar co-monomers (e.g., acrylic acid). The adhesive may have a thickness in the range of from about 0.1 to about 2 mils (2.5 to 50 microns). A release liner may also be employed that contacts the adhesive prior to use. The release liner may contain any of a variety of materials known to those of skill in the art, such as a silicone-coated paper or film substrate.

In addition to being coated onto the cooling product, the thermochromic composition may also be incorporated into one or more substrates of the cooling product. For example, the thermochromic composition may be compounded with a melt-extrudable thermoplastic composition to form a film, fiber, or nonwoven web used in the cooling product. In such embodiments, the thermochromic composition may be pre-blended with a carrier resin to form a masterbatch that is compatible with the thermoplastic composition. Because the thermochromic composition may be more miscible with amorphous regions of a polymer than the crystalline regions, the carrier resin may be generally amorphous or semi-crystalline to optimize compatibility. Exemplary amorphous polymers include polystyrene, polycarbonate, acrylic, acrylonitrile-butadiene-styrene (ABS), styrene-acrylonitrile, and polysulfone. Exemplary semi-crystalline polymers include high and low density polyethylene, polypropylene, polyoxymethylene, poly(vinylidine fluoride), poly(methyl pentene), poly(ethylene-chlorotrifluoroethylene), poly(vinyl fluoride), poly(ethylene oxide), poly(ethylene terephthalate), poly(butylene terephthalate), nylon 6, nylon 66, poly(vinyl alcohol) and polybutene. Particularly desired semi-crystalline polymers are predominantly linear polymers having a regular structure. Examples of semi-crystalline, linear polymers that may be used in the present invention include polyethylene, polypropylene, blends of such polymers and copolymers of such polymers. Semi-crystalline polyethylene-based polymers, for instance, may have a melt index of greater than about 5 grams per 10 minutes, and in some embodiments, greater than about 10 grams per 10 minutes (Condition E at 190° C., 2.16 kg), as well as a density of greater than about 0.910 grams per cubic centimeter (g/cm³), in some embodiments greater than about 0.915 g/cm³, in some embodiments from about 0.915 to about 0.960 g/cm³, in some embodiments from about 0.917 and 0.960 g/cm³. Likewise, semi-crystalline polypropylene-based polymers may have a melt index of greater than about 10 grams per 10 minutes, and in some embodiments, greater than about 20 grams per 10 minutes, as well as a density of from about 0.89 to about 0.90 g/cm³. Specific examples of such polymers include ExxonMobil 3155, Dow polyethylenes such as DOWLEX™ 2517; Dow LLDPE DNDA-1082, Dow LLDPE DNDB-1077, Dow LLDPE 1081, and Dow LLDPE DNDA 7147. In some instances, higher density polymers may be useful, such as Dow HDPE DMDA-8980. Additional resins include Escorene™ LL 5100 and Escorene™ LL 6201 from ExxonMobil. Polypropylene-based resins having a density of from about 0.89 g/cm³ to about 0.90 g/cm³ may also be used, such as homopolymers and random copolymers such as ExxonMobil PP3155, PP1074KN, PP9074MED and Dow 6D43.

The amount of the carrier resin employed will generally depend on a variety of factors, such as the type of carrier resin and thermoplastic composition, the processing conditions, etc. Typically, the carrier resin constitutes from about 10 wt. % to about 80 wt. %, in some embodiments from about 20 wt. % to about 70 wt. %, and in some embodiments, from about 40 wt. % to about 60 wt. % of the masterbatch. The thermochromic substance likewise normally constitutes from about 10 wt. % to about 80 wt. %, in some embodiments from about 20 wt. % to about 70 wt. %, and in some embodiments, from about 40 wt. % to about 60 wt. % of the masterbatch.

The carrier resin may be blended with the thermochromic substance using any known technique, such as batch and/or continuous compounding techniques that employ, for example, a Banbury mixer, Farrel continuous mixer, single screw extruder, twin screw extruder, etc. If desired, the carrier resin and thermochromic substance may be dry blended, i.e., without a solvent. After blending, the masterbatch may be processed immediately or compression molded into pellets for subsequent use. One suitable compression molding device is a die and roller type pellet mill. Specifically, the masterbatch (in granular form) is fed continuously to a pelletizing cavity. The masterbatch is compressed between a die and rollers of the cavity and forced through holes in the die. As pellets of the composition are extruded, a knife or other suitable cutting surface may shear the pellets into the desired size.

Regardless of whether the thermochromic substance is pre-blended with a carrier resin, it may be ultimately compounded with a melt-extrudable thermoplastic composition to form a substrate (e.g., film, fiber, or nonwoven web). The thermochromic substance or masterbatch containing the substance may be miscible with the thermoplastic composition. Otherwise, the components may simply be blended under high shear or modified to improve their interfacial properties. The thermochromic substance may be blended with the thermoplastic composition (e.g., polypropylene or polyethylene) before melt extrusion or within the extrusion apparatus itself. The thermochromic substance may constitute from about 0.001 wt. % to about 10 wt. %, in some embodiments from about 0.01 wt. % to about 5 wt. %, and in some embodiments, from about 0.1 wt. % to about 1 wt. % of the blend.

Exemplary melt-extrudable polymers suitable for use in the thermoplastic composition may include, for example, polyolefins, polyesters, polyamides, polycarbonates, copolymers and blends thereof, etc. Suitable polyolefins include polyethylene, such as high density polyethylene, medium density polyethylene, low density polyethylene, and linear low density polyethylene; polypropylene, such as isotactic polypropylene, atactic polypropylene, and syndiotactic polypropylene; polybutylene, such as poly(1-butene) and poly(2-butene); polypentene, such as poly(1-pentene) and poly(2-pentene); poly(3-methyl-1-pentene); poly(4-methyl-1-pentene); and copolymers and blends thereof. Suitable copolymers include random and block copolymers prepared from two or more different unsaturated olefin monomers, such as ethylene/propylene and ethylene/butylene copolymers. Suitable polyamides include nylon 6, nylon 6/6, nylon 4/6, nylon 11, nylon 12, nylon 6/10, nylon 6/12, nylon 12/12, copolymers of caprolactam and alkylene oxide diamine, etc., as well as blends and copolymers thereof. Suitable polyesters include poly(lactide) and poly(lactic acid)polymers as well as polyethylene terephthalate, polybutylene terephthalate, polytetramethylene terephthalate, polycyclohexylene-1,4-dimethylene terephthalate, and isophthalate copolymers thereof, as well as blends thereof.

If desired, elastomeric polymers may also be used in the thermoplastic composition, such as elastomeric polyesters, elastomeric polyurethanes, elastomeric polyamides, elastomeric polyolefins, elastomeric copolymers, and so forth. Examples of elastomeric copolymers include block copolymers having the general formula A-B-A′ or A-B, wherein A and A′ are each a thermoplastic polymer endblock that contains a styrenic moiety and B is an elastomeric polymer midblock, such as a conjugated diene or a lower alkene polymer. Such copolymers may include, for instance, styrene-isoprene-styrene (S-I-S), styrene-butadiene-styrene (S-B-S), styrene-ethylene-butylene-styrene (S-EB-S), styrene-isoprene (S-I), styrene-butadiene (S-B), and so forth. Commercially available A-B-A′ and A-B-A-B copolymers include several different S-EB-S formulations from Kraton Polymers of Houston, Tex. under the trade designation KRATON®. KRATON® block copolymers are available in several different formulations, a number of which are identified in U.S. Pat. Nos. 4,663,220, 4,323,534, 4,834,738, 5,093,422 and 5,304,599, which are hereby incorporated in their entirety by reference thereto for all purposes. Other commercially available block copolymers include the S-EP-S elastomeric copolymers available from Kuraray Company, Ltd. of Okayama, Japan, under the trade designation SEPTON®. Still other suitable copolymers include the S-I-S and S-B-S elastomeric copolymers available from Dexco Polymers of Houston, Tex. under the trade designation VECTOR®. Also suitable are polymers composed of an A-B-A-B tetrablock copolymer, such as discussed in U.S. Pat. No. 5,332,613 to Taylor, et al., which is incorporated herein in its entirety by reference thereto for all purposes. An example of such a tetrablock copolymer is a styrene-poly(ethylene-propylene)-styrene-poly(ethylene-propylene) (“S-EP-S-EP”) block copolymer.

Examples of elastomeric polyolefins include ultra-low density elastomeric polypropylenes and polyethylenes, such as those produced by “single-site” or “metallocene” catalysis methods. Such elastomeric olefin polymers are commercially available from ExxonMobil Chemical Co. of Houston, Tex. under the trade designations ACHIEVE® (propylene-based), EXACT® (ethylene-based), and EXCEED® (ethylene-based). Elastomeric olefin polymers are also commercially available from DuPont Dow Elastomers, LLC (a joint venture between DuPont and the Dow Chemical Co.) under the trade designation ENGAGE® (ethylene-based) and from Dow Chemical Co. of Midland, Mich. under the name AFFINITY® (ethylene-based). Examples of such polymers are also described in U.S. Pat. Nos. 5,278,272 and 5,272,236 to Lai, et al., which are incorporated herein in their entirety by reference thereto for all purposes. Also useful are certain elastomeric polypropylenes, such as described in U.S. Pat. No. 5,539,056 to Yang et al. and U.S. Pat. No. 5,596,052 to Resconi, et al., which are incorporated herein in their entirety by reference thereto for all purposes.

Once formed, the resulting melt-extrudable blend may then be extruded through a die. Although the die may have any desired configuration, it typically contains a plurality of orifices arranged in one or more rows extending the full width of the machine. The orifices may be circular or noncircular in cross-section. As stated above, the extruded blend may be formed into a thermochromic film in some embodiments of the present invention. Any known technique may be used to form a film from the thermochromic substance, including blowing, casting, flat die extruding, etc. For example, a thermochromic film may be formed by melt extruding the blend, immediately chilling the extruded material (e.g., on a chilled roll) to form a precursor film, and optionally orienting the precursor film in the machine direction, cross machine direction, or both. Alternatively, thermochromic fibers may also be formed according to the present invention. Such fibers may be formed by melt extruding the blend, attenuating the extruded material, and collecting the fibers on a roll (e.g., godet roll) for direct use or on a moving foraminous surface to form a thermochromic nonwoven web.

The thermochromic composition typically constitutes from about 0.5 wt. % to about 25 wt. %, in some embodiments from about 1 wt. % to about 20 wt. %, and in some embodiments, from about 2 wt. % to about 10 wt. % of the dry weight of the cooling product. Of course, the actual amount may vary based on a variety of factors, including the nature of the substrate, sensitivity of the thermochromic substance, the presence of other additives, the desired degree of detectability (e.g., with an unaided eye), etc.

To provide a cooling effect to the desired body part, the cooling product of the present invention generally contains a gel configured to cool the skin of a body part when placed adjacent thereto. The gel may be formed from a crosslinked network that includes one or more gelling polymers. Such polymers may be formed from at least one monomer that is hydrophilic and water-soluble. Some examples of such monomers include, but are not limited to, vinyl pyrrolidone, hydroxyethyl acrylate or methacrylate (e.g., 2-hydroxyethyl methacrylate), hydroxypropyl acrylate or methacrylate, acrylic or methacrylic acid, acrylic or methacrylic esters or vinyl pyridine, acrylamide, vinyl alcohol, ethylene oxide, derivatives thereof, and so forth. Other examples of suitable monomers are described in U.S. Pat. No. 4,499,154 to James, et al., which is incorporated herein in its entirety by reference thereto for all purposes. The resulting polymers may be homopolymers or interpolymers (e.g., copolymer, terpolymer, etc.), and may be nonionic, anionic, cationic, or amphoteric. In addition, the polymer may be of one type (i.e., homogeneous), or mixtures of different polymers may be used (i.e., heterogeneous). In one particular embodiment, the gelling polymer contains a repeating unit having a functional hydroxyl group, such as polyvinyl alcohol (“PVOH”), copolymers of polyvinyl alcohol (e.g., ethylene vinyl alcohol copolymers, methyl methacrylate vinyl alcohol copolymers, etc.), etc. Vinyl alcohol polymers, for instance, have at least two or more vinyl alcohol units in the molecule and may be a homopolymer of vinyl alcohol, or a copolymer containing other monomer units. Vinyl alcohol homopolymers may be obtained by hydrolysis of a vinyl ester polymer, such as vinyl formate, vinyl acetate, vinyl propionate, etc. Vinyl alcohol copolymers may be obtained by hydrolysis of a copolymer of a vinyl ester with an olefin having 2 to 30 carbon atoms, such as ethylene, propylene, 1-butene, etc.; an unsaturated carboxylic acid having 3 to 30 carbon atoms, such as acrylic acid, methacrylic acid, crotonic acid, maleic acid, fumaric acid, etc., or an ester, salt, anhydride or amide thereof; an unsaturated nitrile having 3 to 30 carbon atoms, such as acrylonitrile, methacrylonitrile, etc.; a vinyl ether having 3 to 30 carbon atoms, such as methyl vinyl ether, ethyl vinyl ether, etc.; and so forth.

The degree of hydrolysis may be selected to optimize the cooling properties, solubility, etc., of the polymer. For example, the degree of hydrolysis may be about 90 mole % or greater, in some embodiments about 95 mole % or greater, and in some embodiments, about 99 mole % or more. Such an elevated degree of hydrolysis lowers the solubility of the polymer in water so that it may absorb a greater amount of water to enhance the cooling effect. Examples of suitable highly hydrolyzed polyvinyl alcohol polymers are available under the designation CELVOL™ 165 or 125 from Celanese Corp. If desired, such highly hydrolyzed polyvinyl alcohol polymers may be blended with partially hydrolyzed polymers to improve water solubility. The degree of hydrolysis of such polymers may be less than about 90 mole %, and in some embodiments, from about 85 mole % to about 89 mole %. Examples of suitable partially hydrolyzed polyvinyl alcohol polymers are available under the designation CELVOL™ 203, 205, 502, 504, 508, 513, 518, 523, 530, or 540 from Celanese Corp. When employed, the weight ratio of the partially hydrolyzed to highly hydrolyzed polymers is typically from about 0.1 to about 50, in some embodiments from about 0.5 to about 20, and in some embodiments, from about 1 to about 5. Regardless of the type of polymers employed, however, the concentration of gelling polymer(s) in the gel (based on wt. % of solids) is typically from about 30 wt. % to about 90 wt. %, in some embodiments from about 35 wt. % to about 80 wt. %, and in some embodiments, from about 40 wt. % to about 70 wt. %.

Any known crosslinking technique may be employed, including known ionic or covalent crosslinking techniques. Ionic crosslinking may be induced by contacting the gelling polymer with an ionic crosslinking agent, such as those containing borate, carbonate, sulfate, and other ions. Borate ions, for example, may form strong hydrogen bonds with the hydroxyl groups of the polymer, and thus forms hydrogen-bonded crosslinks between the polymer molecules. Specific examples of suitable ionic crosslinking agents for use in the present invention include sodium tetraborate (e.g., anhydrous sodium tetraborate, sodium tetraborate pentahydrate, sodium tetraborate decahydrate, etc.), potassium tetraborate, sodium carbonate, ammonium sulfate, sodium sulfate, potassium sulfate, aluminum sulfate, zinc sulfate, etc. The concentration of crosslinking agent(s) in the gel (based on wt. % of solids) is typically from about 1 wt. % to about 35 wt. %, in some embodiments from about 5 wt. % to about 30 wt. %, and in some embodiments, from about 10 wt. % to about 25 wt. %.

Although not required, the gel desirably acts as a pressure-sensitive adhesive to improve the self-adhering nature of the cooling product. In this regard, the gel may also contain a plasticizer. Suitable plasticizers may include, for instance, polyhydric alcohol plasticizers, such as sugars (e.g., glucose, sucrose, fructose, raffinose, maltodextrose, galactose, xylose, maltose, lactose, mannose, and erythrose), sugar alcohols (e.g., erythritol, xylitol, malitol, mannitol, glycerol, and sorbitol), polyols (e.g., ethylene glycol, propylene glycol, dipropylene glycol, butylene glycol, and hexane triol), etc. Also suitable are hydrogen bond forming organic compounds which do not have hydroxyl group, including urea and urea derivatives; anhydrides of sugar alcohols such as sorbitan; animal proteins such as gelatin; vegetable proteins such as sunflower protein, soybean proteins, cotton seed proteins; and mixtures thereof. Other suitable plasticizers may include phthalate esters, dimethyl and diethylsuccinate and related esters, glycerol triacetate, glycerol mono and diacetates, glycerol mono, di, and tripropionates, butanoates, stearates, lactic acid esters, citric acid esters, adipic acid esters, stearic acid esters, oleic acid esters, and other acid esters. Aliphatic acids may also be used, such as ethylene acrylic acid, ethylene maleic acid, butadiene acrylic acid, butadiene maleic acid, propylene acrylic acid, propylene maleic acid, and other hydrocarbon based acids. Glycerol is particularly suitable due to its high compatibility with polyvinyl alcohol, high boiling point, and low volatility. The concentration of plasticizer(s) in the gel (based on wt. % of solids) is typically from about 1 wt. % to about 35 wt. %, in some embodiments from about 5 wt. % to about 30 wt. %, and in some embodiments, from about 10 wt. % to about 25 wt. %.

Cooling agents may also be provided in the gel that enhance the physiological cooling sensation to a user. Exemplary cooling agents include menthol, icilin, isopulegol, 3-(1-menthoxy)propane-1,2-diol, 3-(1-menthoxy)-2-methylpropane-1,2-diol, p-menthane-2,3-diol, p-menthane-3,8-diol, 6-isopropyl-9-methyl-1,4-dioxaspiro[4,5]decane-2-methanol, menthyl succinate and its alkaline earth metal salts, trimethylcyclohexanol, N-ethyl-2-isopropyl-5-methylcyclohexanecarboxamide, Japanese mint oil, peppermint oil, menthone, menthone glycerol ketal, menthyl lactate, 3-(1-menthoxy)ethan-1-ol, 3-(1-menthoxy)propan-1-ol, 3-(1-menthoxy)butan-1-ol, 1-menthylacetic acid N-ethylamide, 1-menthyl-4-hydroxypentanoate, 1-menthyl-3-hydroxybutyrate, N,2,3-trimethyl-2-(1-methylethyl )-butanamide, n-ethyl-t-2-c-6 nonadienamide, N,N-dimethyl menthyl succinamide, and menthyl pyrrolidone carboxylate. Other suitable cooling agents are described in 2007/0077331 to Kiefer, et al., which is incorporated herein in its entirety by reference thereto for all purposes. The concentration of cooling agent(s) in the gel (based on wt. % of solids) is typically from about 1 wt. % to about 35 wt. %, in some embodiments from about 5 wt. % to about 30 wt. %, and in some embodiments, from about 10 wt. % to about 25 wt. %.

The gel may also contain a preservative or preservative system to inhibit the growth of microorganisms over an extended period of time. Suitable preservatives for use in the present compositions may include, for instance, alkanols, disodium EDTA (ethylenediamine tetraacetate), EDTA salts, EDTA fatty acid conjugates, isothiazolinone, benzoic esters (parabens) (e.g., methylparaben, propylparaben, butylparaben, ethylparaben, isopropylparaben, isobutylparaben, benzylparaben, sodium methylparaben, and sodium propylparaben), benzoic acid, propylene glycols, sorbates, urea derivatives (e.g., diazolindinyl urea), and so forth. One suitable preservative is Kathon® LX, which is a mixture of methylchloroisothiazolinone and methylisothiazolinone available from Rohm & Haas. Still another suitable preservative is Dowicil 75, which contains 1-(3-chloroallyl)-3,5,7-triaza-1-azoniaadamantane chloride and is available from Dow Chemical Co. of Midland, Mich. The concentration of preservative(s) in the gel (based on wt. % of solids) is typically from about 0.001 wt. % to about 10 wt. %, in some embodiments from about 0.01 wt. % to about 5 wt. %, and in some embodiments, from about 0.1 wt. % to about 4 wt. %.

In addition to the above-mentioned components, other components, such as surfactants, pH adjusters, binders, dyes/pigments/inks, viscosity modifiers, etc., may also be included in the gel of the present invention. When employed, such additional components typically constitute from about 0.001 wt. % to about 10 wt. %, and in some embodiments, from about 0.1 wt. % to about 5 wt. % of the gel.

The gel may be incorporated into the cooling product in a variety of ways. In certain embodiments, for example, the cooling composition may be coated onto a substrate of the cooling product, either alone or in conjunction with a thermochromic composition, using any conventional technique, such as bar, roll, knife, curtain, print (e.g., rotogravure), spray, slot-die, drop-coating, or dip-coating techniques. Upon application, it is normally desired that the resulting gel coating possess a certain moisture content to facilitate its ability to provide cooling. The moisture content is typically from about 60 wt. % to about 99 wt. %, in some embodiments from about 75 wt. % to about 95 wt. %, and in some embodiments, from about 80 wt. % to about 90 wt. %. If desired, the cooling product may be dried to help achieve the desired moisture content. For example, the product may be dried at a temperature of at least about 10° C., in some embodiments at least about 20° C., and in some embodiments, from about 20° C. to about 30° C. The add-on level of the gel may also be varied as desired. The “add-on level” is determined by subtracting the weight of the untreated product from the weight of the treated product, dividing this calculated weight by the weight of the untreated product, and then multiplying by 100%. Lower add-on levels may optimize certain properties (e.g., reduced tackiness), while higher add-on levels may optimize cooling. In some embodiments, for example, the add-on level is from about 200% to about 5000%, in some embodiments from about 500% to about 2500%, and in some embodiments, from about 800% to about 2000%.

Although various configurations of a cooling product have been described above, it should be understood that other configurations are also included within the scope of the present invention. For instance, other layers may also be employed to improve the properties of the cooling product. For example, a first substrate may be employed in conjunction with a second substrate. The substrates may function together to provide cooling to a surface, or may each provide cooling to different surfaces.

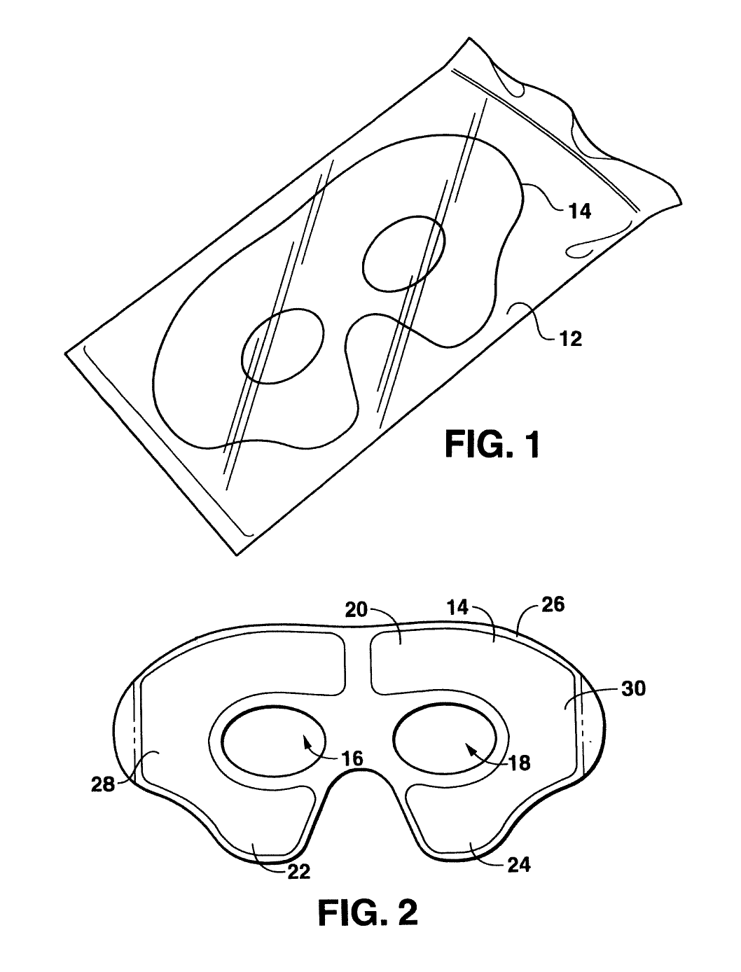

The cooling product may be configured for placement on the skin (e.g., wrapped around) or it may define an interior into which a user may insert a portion of his or her body. Further, the cooling product may have any desired shape or size to accommodate its use on a body part, such as the face, finger, toe, hand, foot, wrist, forearm, etc. Referring to FIG. 1, one embodiment of a cooling product 14 is shown in the shape of a mask sealed within a package 12 that inhibits exposure of the mask 14 to ambient air prior to activation. FIG. 2 illustrates the mask 14 after removal from the package 12. The shape of the mask may depend upon the intended use of the mask. For instance, a mask designed for therapeutic spa-like benefits may have a different shape than a mask used to treat injuries or fevers. In fact, the mask can be designed to cover the entire face, neck and chest of a user. In an alternative embodiment, the mask can be designed to cover a relatively small portion of a person's face. In the embodiment illustrated in FIG. 2, the mask is designed to cover a person's forehead and to surround the eyes and nose of a user. In this regard, the mask 14 includes a first eye opening 16 spaced from a second eye opening 18. The mask 14 further includes a forehead portion 20 located above the eye openings 16 and 18. In addition, the mask 14 includes a pair of lobes that extend downwardly. Specifically, the mask includes a first cheek portion 22 that extends downwardly from the first eye opening 16 and a second cheek portion 24 that extends downwardly from the second eye opening 18. The cheek portions 22 and 24 are designed to surround the nose of a user.

When the mask 14 is designed to treat a person suffering from pain or swelling, it is generally desirable that the mask does not surround the nose of a user so that a user can continue to blow his or her nose even while wearing the mask. For instance, as shown in FIG. 2, the mask 14 may include an access area for the nostrils. In other applications, however, the mask may also include a nose portion that also covers the nose of a user. The nose portion may contain an elastic material, a gathered material that has sufficient slack to go over the nose of a user, or may project outwardly from the mask so that the nose can fit comfortably below the mask. The mask may include a nose portion, for instance, when it is desirable to apply cooling directly to the nose of a user, such as during a spa application or perhaps to provide pain relief when the nose has been injured.

The mask 14 of FIG. 2 also includes a facing layer 26 that supports a first cooling pad 28 and a second cooling pad 30. Although the embodiment in FIG. 2 shows first and second cooling pads 28 and 30, it should be understood that more or less delivery pads may be present. For instance, in one embodiment, the mask may include a single cooling pad that is generally in the shape of the entire mask. The first cooling pad 28 partially encircles the eye opening 16 and thus extends into the forehead portion 20 and down into the first cheek portion 22. Similarly, the second cooling pad 30 partially encircles the second eye opening 18 and also delivers cooling to the forehead portion 20 and to the second cheek portion 24. In this manner, cooling is provided to a user around the eyes, over the forehead, and adjacent to the nose.

The cooling pads 28 and 30 are attached to a facing layer 26 using any known technique, such as thermally bonding, ultrasonically bonding, adhesive bonding, etc. The facing layer 26 may be constructed from nonwoven webs, woven fabrics, knit fabrics, paper webs, etc. Although optional, the mask can further include the outer cover layer 32 to improve the aesthetics and better hold the cooling pads in position. For instance, the outer cover layer can be bonded to the facing layer 26 in a manner that forms pockets for the cooling pads. The outer cover layer 32 has sufficient gas permeability so as not to interfere with the ability of the cooling pad to receive air for gas diffusion. Thus, if present, the outer cover layer can comprise a nonwoven web having a relatively light basis weight and significant porosity.

To hold the mask 14 onto the face of a user, the mask can include a strap (not shown) applied to the facing layer 26. The strap can be made from any suitable material. In one embodiment, for instance, the strap is formed from an elastic material. For instance, the strap can be made from an elastic film or an elastic laminate. In one embodiment, for instance, the strap can be made from a stretch bonded laminate or from a neck bonded laminate. Such materials may provide comfort to the user. Besides a strap, the mask can also include an adhesive for attaching the mask to a person's face.

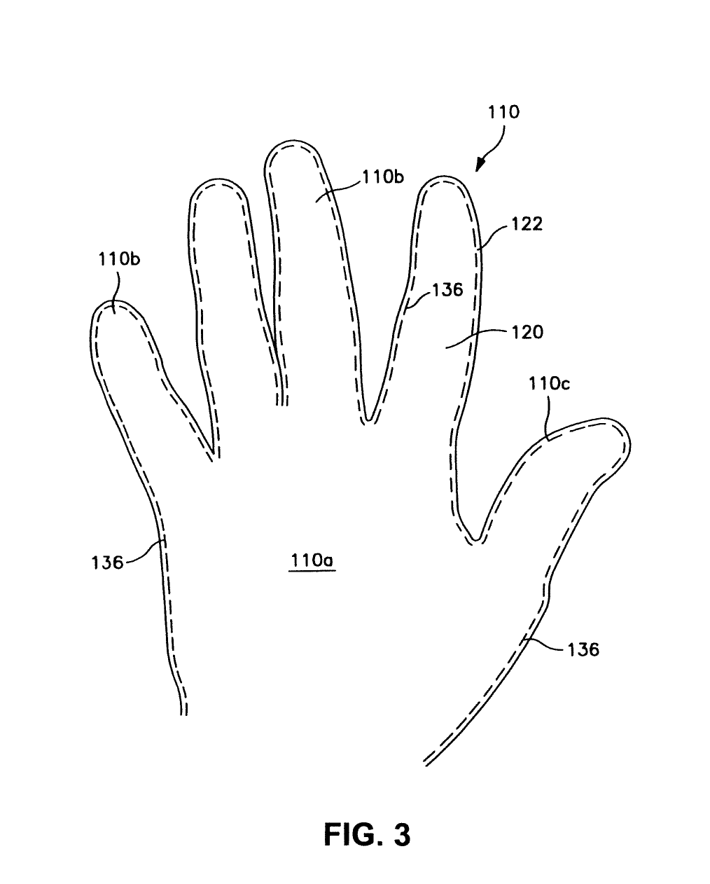

In addition to a mask, the cooling product of the present invention may also have a variety of other configurations, such as gloves, socks, sleeves, mittens, etc. Referring to FIG. 3, for instance, one embodiment of a glove 110 is shown that is in the shape of a human hand. The glove 110 has a palm region 110a, a plurality of finger portions 110b, and a thumb portion 110c. In this particular embodiment, the glove 110 contains substrates 120 and 122 that are joined at a location proximate to their perimeters by sewing and then inverting the glove 110 so that a seam 136 becomes located on the interior of the glove 110. Of course, the glove 110 need not be inverted, and the seam 136 can remain on the exterior of the glove 110. Also, the substrates 120 and 122 need not be joined in a way that produces a seam. For example, the edges of the substrates 120 and 122 may be placed adjacent to each other and joined ultrasonically, thermally, adhesively, cohesively, using tape, by fusing the materials together (e.g., by using an appropriate solvent), by welding the materials together, or by other approaches.

Regardless of the particular configuration of the cooling product, a cooling profile may be achieved in which a reduced temperature is reached quickly and maintained over an extended period of time. For example, a temperature reduction of at least about 1° C., in some embodiments at least about 2° C., and in some embodiments, at least about 3° C., may be achieved in about 1 hour or less, in some embodiments about 30 minutes or less, and in some embodiments, from about 0.1 to about 15 minutes. This may result in a reduced temperature of from about −5° C. to about 45° C., in some embodiments from about 0° C. to about 40° C., and in some embodiments from about 2° C. to about 34° C. This reduced temperature may be substantially maintained for at least about 1 hour, in some embodiments at least about 2 hours, in some embodiments at least about 4 hours, and in some embodiments, at least about 10 hours (e.g., for overnight use). The amount of time that the cooling product remains functional can depend upon the particular application. For instance, when the product is used for aroma therapy or for use in spa-like therapeutic applications, it may only need to be cooled for a period of time of about 15 minutes. When treating a user for pain relief, however, the product may remain cooled for a time of from about 1 hour to about 6 hours, such as from about 2 hours to about 5 hours.

When the cooling product reaches the desired reduced temperature, the thermochromic composition of the present invention may possess one color that indicates to the user that the product is functioning. When the cooling product begins to warm, however, the thermochromic composition undergoes a color change to indicate to the user that the treatment is complete or near completion. This color change is rapid and may be readily detected within a relatively short period of time. For example, a visual change in color may occur in about 30 seconds or less, in some embodiments about 15 seconds or less, and in some embodiments, about 5 seconds or less. Further, the visual color change may remain observable for a sufficient length of time, such as about 1 second or more, in some embodiments about 2 seconds or more, and in some embodiments, from about 3 seconds to about 1 minute. The extent of the color change, which may be determined either visually or using instrumentation (e.g., optical reader), is also generally sufficient to provide a “real-time” indication. This color change may, for example, be represented by a certain change in the absorbance reading as measured using a conventional test known as “CIELAB”, which is discussed in Pocket Guide to Digital Printing by F. Cost, Delmar Publishers, Albany, N.Y. ISBN 0-8273-7592-1 at pages 144 and 145. This method defines three variables, L*, a*, and b*, which correspond to three characteristics of a perceived color based on the opponent theory of color perception. The three variables have the following meaning:

L*=Lightness (or luminosity), ranging from 0 to 100, where 0=dark and 100=light;

a*=Red/green axis, ranging approximately from −100 to 100; positive values are reddish and negative values are greenish; and

b*=Yellow/blue axis, ranging approximately from −100 to 100; positive values are yellowish and negative values are bluish.

Because CIELAB color space is somewhat visually uniform, a single number may be calculated that represents the difference between two colors as perceived by a human. This difference is termed ΔE and calculated by taking the square root of the sum of the squares of the three differences (ΔL*, Δa*, and Δb*) between the two colors. In CIELAB color space, each ΔE unit is approximately equal to a “just noticeable” difference between two colors. CIELAB is therefore a good measure for an objective device-independent color specification system that may be used as a reference color space for the purpose of color management and expression of changes in color. Using this test, color intensities (L*, a*, and b*) may thus be measured using, for instance, a handheld spectrophotometer from Minolta Co. Ltd. of Osaka, Japan (Model #CM2600d). This instrument utilizes the D/8 geometry conforming to CIE No. 15, ISO 7724/1, ASTME1164 and JIS Z8722-1982 (diffused illumination/8-degree viewing system. The D65 light reflected by the specimen surface at an angle of 8 degrees to the normal of the surface is received by the specimen-measuring optical system. Typically, the color change that results is represented by a ΔE of about 2 or more, in some embodiments about 3 or more, and in some embodiments, from about 5 to about 50.

The present invention may be better understood with reference to the following example.

### EXAMPLE

The ability to incorporate a thermochromic composition into a nonwoven fabric for incorporation into a warming product was demonstrated. More specifically, meltblown nonwoven fabrics were made by mixing thermochromic pigment/polypropylene concentrated pellets (colorless to magenta, 11° C. temperature transition, type #15 grid H#8, Chromicolor concentrates, Matsui International Co. Inc., Gardena, Calif.) with meltblown-grade polypropylene pellets (PF-015 Exxon) in a cement mixer for 10 minutes. This effectively blended down the pigment concentration from 18% down to 3.6% wt/wt. The meltblown fabric was spun and the fabric collected without thermal bonding. The following conditions were employed:

| | |
|---|---|
| Extruder melt temperature: | 460° F. |
| Extruder melt pressure: | 260 psi |
| Polymer pipe temperature: | 500° F. |
| Polymer hose temperature: | 500° F. |
| Spin pump temperature: | 500° F. |
| Spin pump pressure: | 502 psi |
| Spin pump speed: | 12 rpms |
| Die melt temperature: | 491° F./522° F. |
| Die pressure: | 70 psi |
| Die primary air temperature: | 700° F. |
| Die primary air pressure at tip: | 2.0 psi |
| Die heater: | 500° F. |
| Picker spped: | 2650 rpms |
| Die height: | 12 inches |
| Pulp nozzle height: | 16 inches |
| Die tip angle: | 45° |
| Line speed: | 225 fpm |

Extruder zone 1=260° F., zone 2=370° F., zone 3=460° F.; zone 4=480° F., zone 5=500° F., zone 6=500° F.

While the invention has been described in detail with respect to the specific embodiments thereof, it will be appreciated that those skilled in the art, upon attaining an understanding of the foregoing, may readily conceive of alterations to, variations of, and equivalents to these embodiments. Accordingly, the scope of the present invention should be assessed as that of the appended claims and any equivalents thereto.

What is claimed is:

1. A cooling product for providing treatment to the body part of a user, the cooling product comprising:
a gel configured to cool the skin of a body part for a certain time period when placed adjacent thereto, the time period defining a cooling cycle;
a thermochromic composition that possesses a first color during the cooling cycle and a second color after completion of the cooling cycle, the first color being visually distinguishable from the second color.

2. The cooling product of claim 1, wherein the thermochromic composition undergoes a color change at a temperature of from about −5° C. to about 45° C.

3. The cooling product of claim 1, wherein the thermochromic composition undergoes a color change at a temperature of from about 2° C. to about 34° C.

4. The cooling product of claim 1, wherein the thermochromic composition includes liquid crystals.

5. The cooling product of claim 1, wherein the thermochromic composition includes microcapsules that contain a proton-accepting chromogen and a desensitizer, wherein the desensitizer possesses a melting point above which the chromogen is capable of becoming protonated, thereby resulting in a color change.

6. The cooling product of claim 5, wherein the proton-accepting chromogen is a leuco dye.

7. The cooling product of claim 5, wherein the desensitizer has a boiling point of about 150° C. or higher and a melting point of about from about 20° C. to about 45° C.

8. The cooling product of claim 5, wherein the microcapsules further comprise a proton-donating agent.

9. The cooling product of claim 1, wherein the cooling product contains a substrate.

10. The cooling product of claim 9, wherein the thermochromic composition is disposed on a surface of the substrate.

11. The cooling product of claim 9, wherein the thermochromic composition is incorporated into the substrate.

12. The cooling product of claim 11, wherein the gel is disposed on a surface of the substrate.

13. The cooling product of claim 9, wherein the substrate contains a nonwoven web.

14. The cooling product of claim 1, wherein the gel is formed from a crosslinked network that includes a gelling polymer.

15. The cooling product of claim 14, wherein the gelling polymer is a vinyl alcohol polymer.

16. The cooling product of claim 1, wherein the cooling product is a mask.

17. The cooling product of claim 1, wherein the cooling product is a glove.

18. A method for monitoring the degree of cooling being provided to a body part of a user, the method comprising:
providing a cooling product that comprises comprising a gel configured to cool the skin of a body part for a certain time period when placed adjacent thereto, 5 the time period defining a cooling cycle, the cooling product further comprising a thermochromic composition that possesses a first color during the cooling cycle and a second color after completion of the cooling cycle;
placing the cooling product adjacent to a body part; and
observing the thermochromic composition, wherein observation of the first 10 color indicates that the gel is cooling the skin during the cooling cycle and observation of the second color indicates that the cooling cycle is complete.

19. The method of claim 18, wherein the thermochromic composition undergoes a color change at a temperature of from about −5° C. to about 45° C.

20. The method of claim 18, wherein the thermochromic composition undergoes a color change at a temperature of from about 2° C. to about 34° C.

21. The method of claim 18, wherein the thermochromic composition includes microcapsules that contain a proton-accepting chromogen and a desensitizer, wherein the desensitizer possesses a melting point above which the chromogen is capable of becoming protonated, thereby resulting in a color change.

22. The method of claim 18, wherein the gel is formed from a crosslinked network that includes a gelling polymer.

23. The method of claim 22, wherein the gelling polymer is a vinyl alcohol polymer.

24. The method of claim 18, wherein the body party is the face.

25. The method of claim 18, wherein the body part is a hand or foot.
